(19) United States
(12) Patent Application Publication
(10) Pub. No.: US 2020/0315534 A1
(43) Pub. Date: Oct. 8, 2020

(54) STRETCHABLE LAMINATED SHEET, METHOD FOR PRODUCING STRETCHABLE LAMINATED SHEET, AND BIOLOGICAL INFORMATION MEASURING GARMENT

(71) Applicant: TOYOBO CO., LTD., Osaka (JP)

(72) Inventors: Haruhiko NARUSAWA, Shiga (JP); Michihiko IRIE, Shiga (JP); Takashi KONDO, Shiga (JP)

(73) Assignee: TOYOBO CO., LTD., Osaka (JP)

(21) Appl. No.: 16/754,425

(22) PCT Filed: Oct. 1, 2018
(86) PCT No.: PCT/JP2018/036679

§ 371 Date: Apr. 8, 2020

(30) Foreign Application Priority Data
Oct. 13, 2017 (JP) 2017-199274

(51) Int. Cl. A61B 5/00

(52) U.S. Cl. CPC A61B 5/6804; A61B 2562/125

(57) ABSTRACT

An object of the present invention is to provide a stretchable laminated sheet which is mainly used for garment for measuring biological information and which is excellent in adhesiveness to a garment, has favorable conductivity and stretchability, has moisture permeability high enough for moisture and sweat to be dissipated, and allows reduction of stuffiness, rash and discomfort. A stretchable laminated sheet of the present invention, brought into contact with skin for measuring a biological signal, comprises a stretchable insulating film layer having adhesiveness and a stretchable film layer having electrical conductivity, wherein the stretchable laminated sheet has a thickness of 10 to 800 μm and a moisture permeability of 300 to 30,000 g/m·24h, and an adhesive strength against peeling at 90 degrees between layers is preferably 0.5 N/cm or more, and the present invention constitutes a garment for measuring biological information having such a sheet as a plurality of skin contact electrodes.

### TECHNICAL FIELD

The present invention relates to a stretchable laminated sheet useful for a biological information measuring garment excellent in comfort and capable of measuring a faint electric signal inside a living body when contacting the skin surface of the living body, a method for producing the stretchable laminated sheet, and a biological information measuring garment including the stretchable laminated sheet.

### BACKGROUND ART

For measuring a faint bioelectric signal inside a living body in electmencephalography, electrocardiography, electromyography or the like, adhesive pad electrodes composed of a soft conductive adhesive gel having conductivity (Patent Document 1) or the like have been heretofore used. When such adhesive pad electrodes are attached on the skin of a subject for a long period of time, moisture or sweat evaporating from the skin may cause stuffiness or rash, resulting in development of dermatitis, which provides a feeling of discomfort. Thus, hydrogel-based adhesive pad electrodes that hardly cause stuffiness or rash have been proposed (Patent Document 2). In addition, stuffiness or rash caused by moisture or sweat evaporating from the skin is regarded as a problem in medical tapes that am attached to the skin as in the case of adhesive pad electrodes, and an effort has been made to use a moisture-permeable support (Patent Document 3) or e a moisture-permeable adhesive (Patent Document 4).

Conventional adhesive pad electrodes enable measurement in a state of resting a subject with independent electrodes attached on the subject. On the other hand, garments on which electrodes am mounted have attracted attention with the demand for measurement of bioelectric signals over a long period of time in daily life, and extensible electrodes have been proposed which can be mounted on garments without risk of discharging a gel and which can closely adhere and adapt to a human body. Examples of the above electrode include electrodes obtained by combining a fabric electrode braided with metal conductor fiber of stainless steel etc. and an electrode partially impermeable to moisture. By making the electrode impermeable to moisture, the sheet resistance between the skin and the electrode is educed by sweating to avoid impacts of electric noises caused by the fabric-based electrode (Patent Document 5). This method may have the problem that since the impedance between the skin and the electrode depends on sweating, stability of acquisition of signals is insufficient when a subject has a dry skin or hardly sweats, or since the electrode is partially impermeable, moisture or sweat evaporating from the skin may cause stuffiness or rash, resulting in development of dermatitis.

In addition, a sheet-like electrode formed by drying a conductive paste containing resin, conductive metal powder and an organic solvent is capable of stably acquiring signals because the entire surface of the electrode closely adheres to and follows the skin, but since the sheet-like electrode is impermeable to moisture, moisture or sweat evaporating from the skin may cause stuffiness or rash, resulting in development of dermatitis or impairment of a wear feeling (Patent Document 6).

### RELATED ART DOCUMENTS

### Patent Documents

### Patent Document 1: JP-A-09-215668

### Patent Document 2: WO2016/080082

### Patent Document 3: JP-A-2001-161741

### Patent Document 4: JP-A-2009-233316

### Patent Document 5: JP-A-2006-512128

### Patent Document 6: WO2016/114339

### DISCLOSURE OF THE INVENTION

### Problems To be Solved by the Invention

Thus, regarding a garment on which electrodes are mounted with the aim of measuring electric signals over a long period of time in daily life, it is necessary that the electrodes closely adhere to and follow the skin to prevent electric noises and degradation of electric signals even when the garment turns from a dried state into a state of being wet with in or sweat or a wearer makes various postures or motions in daily life. In addition, the garment is worn full-time, and therefore in view of maintenance of a feeling of fitting, reduction of a feeling of shrinking, and reduction of causes and discomfort of dermatitis due to stuffiness and rash in comfort of the garment, the electrode is required to have air permeability and moisture permeability, and have stretchability and flexibility while adhering to the garment without easily moving.

On the other hand, at the time when a stretchable electrode composed of a resin and conductive fine particles is formed into a sheet, a liquid material as a precursor containing an organic solvent and a monomer is applied or printed, and then dried or cured to form the electrode into a sheet. At this time, the resin shrinks due to volatilization of the organic solvent or curing of the resin, and therefore the conductive fine particles are densely linked to obtain favorable conductivity, and at the same time, voids which are not filled with the resin may be formed in gaps of the chain structure of the conductive fine particles, so that air permeability or moisture permeability can be obtained. In the case where when the conductive sheet has air permeability or moisture permeability as described above, an adhesive resin used for mounting the conductive sheet on the garment does not have air permeability or moisture permeability, or where when an adhesive resin having air permeability or moisture permeability is used, voids of the conductive sheet is filled with the adhesive resin, needed moisture permeability cannot be obtained.

The present invention has been made in view of the problems of conventional arts, and an object of the present invention is to provide a stretchable laminated sheet which is excellent in adhesiveness to a garment, has favorable conductivity and stretchability, has moisture permeability high enough for moisture and sweat to be dissipated, and allows reduction of stuffiness, rash and discomfort.

### Solutions to the Problems

That is, the present invention includes the following configurations.

[1] A stretchable laminated sheet, brought into contact with skin for measuring a biological signal, comprising:

a stretchable insulating film layer having adhesiveness; and

a stretchable film layer having electrical conductivity, wherein

the stretchable laminated sheet has a thickness of 10 to 800 μm, and

the stretchable laminated sheet has a moisture permeability of 300 to 30,000 g/m²·24 h.

[2] The stretchable laminated sheet according to (1), wherein

the stretchable laminated sheet has a tensile elastic modulus of 500 MPa or less, and

a load applied to the stretchable laminated sheet at an extension ratio of 10% is 100 N or less.

[3] The stretchable laminated sheet according to [1] or [2], wherein

the stretchable film layer having electrical conductivity has a sheet resistance of 300 Ω_{□} or less in the absence of extension, and

the stretchable film layer having electrical conductivity has a sheet resistance increase ratio of less than 10 at an extension ratio of 10%

[4] The stretchable laminated sheet according to any one of [1] to [3], wherein

an adhesive strength against peeling at 90 degrees between the stretchable insulating film layer having adhesiveness and the stretchable film layer having electrical conductivity is 0.5 N/cm or more.

[5] The stretchable laminated sheet according to any one of [1] to [4], wherein

the stretchable film layer having electrical conductivity includes a conductive film including conductive fine particles and a binder resin.

[6] The stretchable laminated sheet according to anyone of [1] to [5], wherein

the binder resin has an elastic modulus of 1 GPa or less and

the binder resin has a rupture elongation of 200% or more.

[7] A method for producing the stretchable laminated sheet according to anyone of [1] to [6], comprising

forming the stretchable film layer having electrical conductivity by a coating method or a printing method.

[8] A biological information measuring garment comprising

a plurality of body contact components including the stretchable laminated sheet according to any one of [1] to [6].

Further, the present invention preferably includes the following configuration.

[9] The stretchable laminated sheet according to anyone of [1] to [6], wherein an adhesive strength against peeling at 90 degrees is 0.5 N/cm or more and 20 N/cm or less, wherein a pressure-sensitive adhesive tape including a base material having a tensile elastic modulus of 1 GPa or more and a thickness of 20 μm or more was attached to each of the stretchable insulating film layer having adhesiveness and the stretchable film layer having electrical conductivity in the stretchable laminated sheet, and each of the layers was held together with the pressure-sensitive adhesive tape attached to each of the layers, and on the basis of JIS K6854-1(1999), “Adhesive-Method for Testing Adhesive Strength against Peeling-Peeling at 90 Degrees”, the interlayer peeling strength between the stretchable insulating film layer having adhesiveness and the stretchable film layer having electrical conductivity was measured as the adhesive strength against peeling at 90 degrees of the stretchable laminated sheet.

### Effects of the Invention

In the present invention, the stretchable laminated sheet which is attached to part of the inside of a garment to contact the skin for measuring biological signals includes at least two layers: a stretchable insulating film layer having adhesiveness and a stretchable film layer having electrical conductivity. The stretchable insulating film layer having adhesiveness plays a role to bond a garment or the like to the stretchable film layer having electrical conductivity, and prevent rupture of the chain structure of conductive fine particles in the stretchable film layer having electrical conductivity at extension, so that high conductivity is maintained even when the sheet is repeatedly stretched.

Further, when the thickness of the stretchable laminated sheet is 10 to 800 μm and the moisture permeability of the stretchable laminated sheet is 300 to 30,000 g/m²·24 h, the stretchable film layer having electrical conductivity closely adheres to the skin to obtain stable electric signals, and dissipates moisture and sweat evaporating from the skin, so that stuffiness, rash and discomfort are reduced. In addition, since the tensile elastic modulus of the stretchable laminated sheet is 500 MPa or less, and the load at extension is 100 N or less when the extension ratio of the stretchable laminated sheet is 10%, the stretchable laminated sheet follows a fabric which is deformed at the time of changing a posture for putting on the garment, so that a sense of discomfort does not occur. Thus, the stretchable laminated sheet is flexible and stretchable, and therefore does not impair a feeling of wearing when mounted on the garment.

In addition, the stretchable film layer having electrical conductivity has a sheet resistance of 300 Ω_{□} or less in the absence of extension, and a sheet resistance increase ratio of less than 10 at an extension ratio of 10%. Therefore electric signals necessary for measurement am obtained, and stable electric signals am secured even when the stretchable film layer having electrical conductivity is extended along with a fabric that is deformed when the posture is changed at the time of wearing. The adhesive strength against peeling at 90 degrees between the stretchable insulating film layer having adhesiveness and the stretchable film layer having electrical conductivity is 0.5 N/cm or more, and therefore stretchable film layer having electrical conductivity is not peeled at the time of use or washing. The upper limit of the adhesive strength against peeling at 90 degrees is preferably about 15N/cm.

The stretchable film layer having electrical conductivity includes conductive fine particles and a binder resin. By using the conductive fine particles, electrical signals are secured, and the fine particles are fixed with the binder resin to maintain a shape as a stretchable film layer having electrical conductivity. Here, by using a binder resin having an elastic modulus of 1 GPa or less and a fracture elongation of 200 or more, a stretchable film layer having excellent stretchability and electrical conductivity is obtained, the stretchable film layer having electrical conductivity follows a fabric, so that a sense of discomfort does not occur, and thus stable electric signals are secured even when the stretchable film layer having electrical conductivity is extended along with the fabric.

### Mode for Carrying Out the Invention

Hereinafter, a stretchable laminated sheet according to an embodiment of the present invention will be described. The stretchable laminated sheet of the present invention has a sheet shape with a thickness of 10 to 800 μm, has a moisture permeability of 300 to 30,000 g/m²·24 h, and is mounted on pat of the inside of a garment where a stretchable insulating film layer having adhesiveness is interposed therebetween. When the thickness is less than 10 μm, the electric resistance increases, and the stretchable film layer having electrical conductivity may be broken and insulated when the sheet is deformed at extension. On the other hand, when the thickness is mom than 800 μm, the load increases at extension, so that when a fabric is extended, the stretchable laminated sheet does not follow the fabric, and a level difference is generated between the sheet and the fabric, leading to impairment of a feeling of wearing. When the moisture permeability is less than 300 g/m²·24 h, ventilation at the sheet part is hindered, so that moisture or sweat evaporating from the skin may cause stuffiness or rash, resulting in development of dermatitis or impairment of a feeling of wearing, and when the moisture permeability is mom than 30,000 g/m²·24 h, the electric resistance of the stretchable film layer having electrical conductivity increases due to excessiveness of voids, so that stable electrical signals cannot be obtained, and the physical properties of the film layer are deteriorated. The stretchable laminated sheet of the present invention includes at least two layers: an insulating stretchable film layer having adhesiveness and a stretchable film layer having electrical conductivity. When the sheet is used as wiring, a stretchable insulating film layer may be further provided on the front side of the stretchable film layer having electrical conductivity

The stretchable insulating film layer having adhesiveness is made of flexible resin, examples thereof include thermoplastic resins, thermosetting resins or photo-curable resins, and rubbers or elastomers. Examples of the thermoplastic resin include low-density polyethylene, ethylene-vinyl acetate copolymers, polyvinylidene chloride and copolymer polyester. Examples of the thermosetting resin or photo-curable resin include acrylic resins, silicon resins and polyurethane resins. Examples of the rubber or elastomer include urethane rubber, acrylic rubber, silicone rubber, butadiene rubber, nitrile rubber, isoprene rubber, styrene butadiene rubber, butyl rubber, chlorosulfonated polyethylene rubber, ethylene propylene rubber and vinylidene fluoride copolymer. For the layer, it is preferable to use rubber/elastomer in order to develop adhesiveness and stretchability. Further, for developing moisture permeability, it is preferable to use a flexible resin containing a hydrophilic component or a hydrophilic resin, or a microporous stretchable film prepared from a flexible resin by a wet film formation method or draw film formation method. The flexible resin may contain water or an organic solvent for coating or printing.

The stretchable film layer having electrical conductivity includes at least two or more kinds of components: conductive fine particles and a binder resin, and may each have two or more layers different in chain structure of components and conductive fine particles. The conductive fine particles are metal-based fine particles and/or carbon-based fine particles. Examples of the metal-based fine particles include metal particles such as particles of silver, gold, platinum, palladium, copper, nickel, aluminum, zinc, lead and tin; alloy particles such as particles of brass, bronze, white copper and solder; hybrid particles such as particles of silver-coated copper; metal-plated polymer particles, metal-plated glass particles, and metal-coated ceramic particles. Examples of the carbon-based fine particles include graphite powder, activated carbon powder, scaly graphite powder, acetylene black, ketjen black, fullerene and carbon nanotube. The conductive fine particles may be of only one type or of two or more types.

The binder resin is preferably a resin having an elastic modulus of 1 GPa or less and a rupture elongation of 200% or more, and examples thereof include thermoplastic resins, thermosetting resins or photo-curable resins, and rubbers or elastomers. Examples of the thermoplastic resin include low-density polyethylene, ethylene-vinyl acetate copolymers, polyvinylidene chloride and copolymer polyester. Examples of the thermosetting resin or photocurable resin include acrylic resins, silicon resins and polyurethane resins. Examples of the rubber or elastomer include urethane rubber acrylic rubber, silicone rubber, butadiene rubber, nitrile rubber, isoprene rubber, styrene butadiene rubber, butyl rubber, chlorosulfonated polyethylene rubber, ethylene propylene rubber and vinylidene fluoride copolymer. Only one binder resin may be used, or two or more binder resins may be used. For the layer, it is preferable to use rubber/elastomer in order to develop adhesiveness with conductive fine particles and stretchability.

The blending amount of the conductive fine particles is determined with consideration given to electric resistance, stretchability and moisture permeability. When the volume ratio (%) to the binder resin is large, electric resistance decreases, leading to suppression of degradation of electric signals, and the number of voids in the film layer increases, leading to moisture permeability, but washing resistance and stretchability are reduced, leading to deterioration of a feeling of shrinking and the feeling of fitting. On the other hand, when the volume ratio (%) is small, stretchability increases, leading to improvement of shrinking and the feeling of fitting, but the number of voids in the film layer decreases, so that moisture permeability is reduced, and thus electrical resistance increases, leading to degradation of electric signals. For balancing both the properties of the conductive fine particles and the binder resin, the blending amount of the conductive fine particles based on the amount of the binder resin is preferably 20 to 60% by volume.

As long as necessary properties am not impaired, one or both of the stretchable insulating film layer having adhesiveness and the stretchable film layer having electrical conductivity in the stretchable laminated sheet of the present invention may contain insulating fine particles for obtaining mechanical properties, heat resistance, durability and moisture permeability. The insulating fine particles are fine particles composed of an organic or inorganic insulating substance. Examples of the organic fine particles include resin-based fine particles such as acrylic resin fine particles, styrene resin fine particles and melamine resin fine particles. Examples of the inorganic fine particles include ceramic-based fine particles such as those of silica, alumina, zirconia, talc, silicon carbide, magnesia and boron nitride; and fine particles of salts hardly soluble in water such as calcium phosphate, magnesium phosphate, barium sulfate and calcium sulfate. The insulating fine particles may be of only one type or of two or more types.

The stretchable film layer having electrical conductivity of the present invention is prepared by application or printing of a conductive paste containing water or an organic solvent in addition to the conductive fine particles and the binder resin. The content of the water or organic solvent depends on the method for dispersing the conductive fine particles, and on the viscosity of a conductive paste suitable for the method for forming a conductive film and on the method for drying the conductive paste, or the like, and affects the chain structure shape form of the conductive fine particles and the moisture permeability of the film layer after application of the conductive paste. The content of the water or organic solvent is not particularly limited, and is preferably 20 to 60% where the total mass of the conductive fine particles, the binder resin and the solvent is defined as 100%. When the content of the water or organic solvent is less than 20% by mass, the conductive paste has a high viscosity, and is thus unsuitable for application or printing, the chain structure of the conductive fine particles is nonuniform, leading to reduction of conductivity and stretchability, and the void ratio in the film decreases, leading to reduction of moisture permeability. On the other hand, when the content of the water or organic solvent is more than 60% by mass, the amount of a residual solvent in the dry-cured coating film may increases, resulting in deterioration of reliability of the coating film.

The boiling point of the organic solvent is preferably 100° C. or higher and lower than 300° C. When the boiling point of the organic solvent is excessively low, there is a risk of volatilizing the solvent in the paste production process or at the time of use of the paste, so that the ratio of components forming the paste is easily changed, and when the boiling point of the organic solvent is excessively high, the amount of a residual solvent may increase, resulting in deterioration of reliability of the coating film. Examples of the organic solvent include cyclohexanone, toluene, isophorone, γ-butyrolactone, benzyl alcohol, Solvesso 100, 150 and 200 manufactured by Exxon Chemical, propylene glycol monomethyl ether acetate, terpineol, butyl glycol acetate, diamylbenzene (boiling point 260 to 280° C.), triamylbenzene (boiling point 300 to 320° C.), n-dodecanol (boiling point 255 to 29° C.), diethylene glycol (boiling point 245° C.), ethylene glycol monoethyl ether acetate (boiling point: 145° C.), diethylene glycol monoethyl ether acetate (boiling point: 217° C.), diethylene glycol monobutyl ether acetate (boiling point 247), diethylene glycol dibutyl ether (boiling point: 255° c.), diethylene glycol monoacetate (boiling point: 250° C.), triethylene glycol diacetate (boiling point: 300° C.) triethylene glycol(boiling point: 276° C.), triethylene glycol monomethyl ether(boiling point: 249° C.), triethylene glycol monoethyl ether(boiling point 256° C.), triethylene glycol monobutyl ether (boiling point 271° C.), tetraethylene glycol (boiling point: 327° C.), tetraethylene glycol monobutyl ether (boiling point 304° C.), tripropylene glycol (boiling point: 267° C.), tripropylene glycol monomethyl ether (boiling point: 243° C.), and 2,2,4-trimethyl-,3-pentanediol monoisobutyrate (boiling point:253° C.), and if necessary, two or more thereof may be contained. Such organic solvent is appropriately contained so that the conductive paste has a viscosity suitable for application or printing.

In addition, as long as necessary properties for a stretchable conductive film are not impaired, a conductive paste to be used for preparing the stretchable film layer having electrical conductivity of the present invention may contain a thixotropy imparting agent, a leveling agent, a plasticizer, a defoaming agent and the like for obtaining application and printing properties. Using the conductive paste for forming the stretchable film layer having electrical conductivity according to the present invention enables conductive fine particles to be uniformly dispersed in a resin by using a previously known method for dispersing fine particles in a liquid. For example, after a dispersion liquid of fine particles and a resin solution can be mixed, followed by uniformly dispersing the fine particles by an ultrasonic method, a mixer method, a three-roll mill method, a ball mill method or the like. Two or mom of these methods can be used in combination.

The method for producing a stretchable laminated sheet according to the present invention is preferably one of a method in which a conductive paste is applied or printed onto a releasable base material to form a coating film, an organic solvent contained in the coating film is then volatilized to perform drying, and a preformed stretchable insulating film layer sheet having adhesiveness is then laminated to the coating film; a method in which a coating film is formed by applying or printing a conductive paste to a preformed stretchable insulating film layer sheet having adhesiveness, and an organic solvent contained in the coating film is then volatilized to perform drying. On the other hand, as compared to a method in which a conductive paste is applied or printed onto a releasable base material to form a coating film, an organic solvent contained in the coating film is then volatilized to perform drying, and a stretchable insulating film layer having adhesiveness is then formed thereon, a method in which a flexible resin solution is applied or printed to form a coating film, and an organic solvent contained in the coating film is volatilized to perform drying is not preferable because the flexible resin may penetrate into voids of the flexible film layer having electronic conductivity, leading to reduction of moisture permeability.

The step of applying a conductive paste is not specifically limited, and for example, a coating method or a printing method can be used. Examples of the printing method include a semen printing method, a lithographic offset printing method, an inkjet method, a flexographic printing method, a gravure printing method, a gravure offset printing method, a stamping method, a dispensing method and squeegee printing. The step of heating and drying the applied conductive paste or flexible resin solution can be carried out in the air, a vacuum atmosphere, an inert gas atmosphere, a reducing gas atmosphere or the like, the organic solvent is volatilized, curing reaction may proceed under heating, the stretchable film layer having electrical conductivity has favorable electric resistance and stretchability after drying. The heating temperature in the air is in the range of 50 to 200° C., and the heating time is in the range of 10 to 90 minutes. A combination of the heating temperature and the heating time is selected from combinations of a low temperature and a long time and combinations of a high temperature and a short time with consideration given to the electric resistance and stretchability of the stretchable film layer having electrical conductivity, the heat resistance of the binder resin, the boiling point of the organic solvent, and the like. When the temperature is lower than 50° C. and the time is less than 10 minutes the solvent remains in the coating film, so that desired electric resistance or stretchability cannot be obtained. When the temperature is higher than 200° C. and the time is mom than 90 minutes, the binder resin or flexible resin or the base material is degraded, so that desired stretchability cannot be obtained, and costs are increased.

A biological information measuring garment of the present invention has a configuration in which the stretchable laminated sheet of the present invention is mounted on part of the inside of a garment. The base material of the biological information measuring garment of the present invention is not particularly limited as long as it is a strip-shaped material such as a belt or a brassiere, and/or a garment composed of a knitted or woven fabric or a nonwoven fabric, and a stretchable garment is preferable from the viewpoint of the fitting property to a body in wearing, followability in exercise and movement, or the like for measuring biological information. Such a biological information measuring garment serves as means for measuring the biological information of a wearer, ensures a normal wearing method and wear feeling, and enables various kinds of biological information to be conveniently measured only by wearing the garment.

### EXAMPLES

Specific examples of the present invention will now be described, which should not be construed as particularly limiting the present invention.

[Preparation of Conductive Pastel]

Using the materials shown in Table 1, the resin was dissolved in each solvent at a weight ratio as shown in Table 2, conductive fine particles were added into the resulting solution, and the mixture was stirred with a three-roll mill to obtain a conductive paste.

TABLE 1

| Materials | Abbreviation | Substances |
|---|---|---|
| resin | CSM | chlorosulfonated polyethylene rubber CSM-TS530 manufactured by Tosoh Corporation |
| | DN3 | nitrile rubber Nipol DN003 manufactured by Zeon Corporation |
| | UR6 | polyester-urethane resin Vyron UR6100 manufactured by Toyobo Co., Ltd. |
| solvent | CHX | cyclohexanone (boiling point: 156° C.) |
| | IPH | isophorone (boiling point 215° C.) |
| | SOL | solvesso#150 (boiling point: 179 to 203° C.) |
| conductive | G35 | aggregated silver powder G-35 manufactured by DOWA Electronics Materials Co., Ltd. |
| fine particles | FA3 | flake-formed silver powder FA-D-3 manufactured by DOWA Electronics Materials Co., Ltd. |
| adhesive | H120 | polyurethane resin for moisture permeable waterproof bonding manufactured by Sanyo Chemical Industries, Ltd. |
| | | Sanpuren H-120 (non-volatile content 45% by weight, dimethylformamide solution) |
| resin solution | W600 | polyurethane resin for packaging gravure ink manufactured by Arakawa Chemical Industries, Ltd. |
| | | Yuriano W600 (non-volatile content 35% by weight, water/isopropyl alcohol solution) |

TABLE 2

| item | | PS01 | PS02 | PS03 |
|---|---|---|---|---|
| resin | CSM | 20 | — | — |
| [parts by mass] | DN3 | — | 20 | — |
| | UR6 | — | — | 20 |
| solvent | CHX | — | — | 25 |
| [parts by mass] | IPH | — | 45 | — |
| | SOL | 45 | — | 20 |
| silver particles | G35 | 80 | 60 | 60 |
| [parts by mass] | FAD3 | — | 20 | 20 |

[Production of Stretchable Laminated Sheet]

Lamination method 1: The conductive paste was applied onto a release-treated PET film with an applicator in such a manner that the dry thickness was about 60 μm, and drying was performed in a hot air drying oven at 120° C. for 30 minutes to form a stretchable film layer having electrical conductivity. Similarly, an adhesive resin solution as shown in Table 1 was applied onto a separately prepared release-treated PET film with an applicator in such a manner that the dry thickness was about 60 μm, and drying was performed in a hot air drying oven at 120° C. for 30 minutes to form a stretchable insulating film layer having adhesiveness. Next, the stretchable film layer provided with a release-treated PET film and having electrical conductivity and the stretchable insulating film layer provided with a release-treated PET film and having adhesiveness were superposed on each other in such a manner that the release-treated PET film was on the outer side, and using a roll laminator with a rubber roll temperature adjusted to 120° C., both the layers were bonded together to prepared a stretchable laminated sheet provided with a PET film release-treated on both sides.

Lamination method 2: The conductive paste was applied onto a release-treated PET film with an applicator in such a manner that the dry thickness was about 60 μm, and drying was performed in a hot air drying oven at 120° C. for 30 minutes to form a stretchable film layer having electrical conductivity. Next, an adhesive resin solution as shown in Table 1 was applied with an applicator so as to cover the formed stretchable film layer having electrical conductivity in such a manner that the dry thickness was about 60 μm, and drying was performed in a hot air drying oven at 120° C. for 30 minutes to form a stretchable laminated sheet provided with a PET film release-treated at one side.

Using the sheet, the thickness, the moisture permeability, the tensile elastic modulus, the unit load at extension by 10%, the film electric resistance at non-extension, the resistance increase ratio at extension by 10% elongation, and the adhesive strength against peeling at 90 degrees were measured by the methods described below. The measurement results in examples are shown in Table 3.

TABLE 3

| | | | | | Comparative | Comparative | Comparative | Comparative |
|---|---|---|---|---|---|---|---|---|
| | | unit | Example 1 | Example 2 | Example 3 | Example 1 | Example 2 | Example 3 |
| conductive paste | | — | PS1 | PS2 | PS3 | PS1 | PS2 | PS3 |
| adhesive resin | | — | H120 | H120 | W600 | H120 | W800 | W600 |
| lamination method | | — | 1 | 1 | 1 | 2 | 2 | 2 |
| sheet properties | thickness | μm | 126 | 123 | 118 | 116 | 112 | 114 |
| | moisture | g [symbol] m [symbol] · 24 h | 1300 | 1500 | 900 | 270 | 220 | 180 |
| | permeability | | | | | | | |
| | tensile | mPa | 120 | 135 | 150 | 120 | 150 | 135 |
| | elastic | | | | | | | |
| | modulus | | | | | | | |
| | unit load | N/cm | 72 | 81 | 86 | 69 | 84 | 76 |
| | at an | | | | | | | |
| | extension | | | | | | | |
| | ratio of 10% | | | | | | | |
| | film | [symbol] | 161 | 123 | 144 | 182 | 136 | 196 |
| | electric | | | | | | | |
| | resistance | | | | | | | |
| | at non-extension | | | | | | | |
| | sheet resistance | — | 1.9 | 2.5 | 2.3 | 2.1 | 1.9 | 2.2 |
| | increase ratio | | | | | | | |
| | at an extension | | | | | | | |
| | ratio of 10% | | | | | | | |
| | adhesive strength | N/cm | 1.5 | 1.3 | 0.9 | 2.9 | 2.4 | 1.3 |
| | against peeling | | | | | | | |
| | at 90 degrees | | | | | | | |
| skin irritation | skin irritation | — | 0.0 | 0.0 | 2.2 | 6.8 | 6.8 | 9.1 |
| | index | | | | | | | |
| | safety | — | safe product | safe product | safe product | acceptable | acceptable | acceptable |
| | evaluation | | | | | product | product | product |

[symbol]

indicates data missing or illegible when filed

[Measurement of Thickness, Elastic Modulus and Load at Extension by 10%]

The thickness was measured on the basis of JIS K7130 (1999) “Plastics & #8722; Method for Measurement of Film and Sheet, Method A”. The elastic modulus and the load at extension were measured on the basis of JIS K7161-1 (2012), “Plastics: Method for Determination of Tensile Properties-Part 1”. The measurement conditions were set to a test piece width of 15 mm, a test piece length of 150 mm, a chuck-to-chuck distance of 100 mm and a test speed of 50 mm/min.

[Measurement of Moisture Permeability]

The moisture permeability of the stretchable laminated sheet was measured on the basis of JIS L1099 (2012) “Moisture Permeability Test Method for Textile Products, A-1 Method (Calcium Chloride Method)”.

[Extension Test and Measurement of Electric Resistance]

In an extension tester (hand-drawing stretching machine) provided with two 2.5 cm-wide chucks, the stretchable laminated sheet obtained above was held between the chucks with the chuck-to-chuck distance set to 5.0 cm, and the test piece was extended to an extension ratio of 10% in a longitudinal direction (displacement amount: 0.5 cm). Using a digital multimeter (“YOKOGAWA TY530” manufactured by YOKOGAWA Meters & Instruments Corporation) resistance values (Ω) before and after extension were measured outside the two opposed chucks (measurement distance: 10 cm) to obtain electric resistance (Ω□) as resistance before and after the test. The resistance value was measured immediately after extension (3 seconds or less after extension).

[Measurement of Adhesive Strength Against Peeling at 90 Degrees]

A pressure-sensitive adhesive tape including a base material having a tensile elastic modulus of 1 GPa or more and a thickness of 20 μm or more was attached to each of the stretchable insulating film layer having adhesiveness and the stretchable film layer having electrical conductivity in the stretchable laminated sheet, and each of the layers was held together with the pressure-sensitive adhesive tape attached to each of the layers. On the basis of S K6854-1(1999), “Adhesive-Method for Testing Adhesive Strength against Peeling-Peeling at 90 Degrees”, the interlayer peeling strength between the stretchable insulating film layer having adhesiveness and the stretchable film layer having electrical conductivity was measured as the adhesive strength against peeling at 90 degrees of the stretchable laminated sheet. The measurement conditions were set to a test piece width of 15 mm, a test piece length of 150 mm and a peeling speed of 50 mm/min.

In this example, a pressure-sensitive adhesive tape having a 25 μm-thick pressure-sensitive adhesive layer provided on a polyester base material having a thickness of 0 μm was used.

[Primary Irritation Test on Skin]

On the basis of SEK 48-Hour Human Closed Patch Testing, the following primary irritation test on skin was conducted.

The stretchable electrode was cut to a 0.8 cm square, and applied to the back of each of a total of 22 Japanese male and female subjects, and an adhesive bandage for patch testing was attached onto the stretchable electrode. 30 to 60 minutes after the adhesive bandage was removed 48 hours after attachment and about 24 hours after the adhesive bandage was removed 72 hours after attachment, a skin symptom was visually examined to perform evaluation.

The evaluation criteria were set as follows: score 0.0 for no response; score 0.5 for faint erythema; score 1.0 for clear erythema, score 2.0 for erythema and edema or papule; score 3.0 for erythema and edema/papule and small blisters; and score 4.0 for large blisters. A score for each subject was determined, and a skin irritation index was determined from the following formula (1).

Skin irritation index=total score/number of subjects×100 (1)

Further, on the basis of the determined skin irritation index, safety was evaluated from Skin Irritation Index Classification of Cosmetic Products in FY 1995 (safe product: 5.0 or less; acceptable product 5.0 to 15.0; improvement-needed product: 15.0 to 30.0; and dangerous product 30.0 or more)

As described above, the stretchable laminated sheet of the present invention has moisture permeability, and therefore even when the sheet is used in a state of closely contacting a human body, moisture and sweat evaporating from the skin are dissipated, so that stuffiness, rash and discomfort are reduced. In addition, the stretchable film layer having electrical conductivity is not peeled at the time of use or washing because the stretchable laminated sheet has adhesiveness, the wear comfort and the feeling of wearing am not impaired because the load at extension is low, and biological electric signals with less electric noises can be obtained because increase in electric resistance is suppressed at extension.

### INDUSTRIAL APPLICABILITY

The present invention provides a stretchable laminated sheet and a garment for measurement biological information, which hardly cause development of skin rash and skin inflammation, are excellent in wear comfort and feeling of wearing, and enables favorable electric signal measurement. The stretchable laminated sheet and the garment measurement can be applied to health management in everyday life, grasping of biological information in outdoor sports such as jogging and marathon, and labor management in outdoor work at building sites.

1. A stretchable laminated sheet, brought into contact with skin for measuring a biological signal, comprising:
a stretchable insulating film layer having adhesiveness; and
a stretchable film layer having electrical conductivity, wherein
the stretchable laminated sheet has a thickness of 10 to 800 μm, and
the stretchable laminated sheet has a moisture permeability of 300 to 30,000 g/m²·24 h.

2. The stretchable laminated sheet according to claim 1, wherein
the stretchable laminated sheet has a tensile elastic modulus of 500 MPa or less, and
a load applied to the stretchable laminated sheet at an extension ratio of 10% is 100 N or less.

3. The stretchable laminated sheet according to claim 1, wherein
the stretchable film layer having electrical conductivity has a sheet resistance of 300 Ω_{□} or less in the absence of extension, and
the stretchable film layer having electrical conductivity has a sheet resistance increase ratio of less than 10 at an extension ratio of 10%.

4. The stretchable laminated sheet according to claim 1, wherein
an adhesive strength against peeling at 90 degrees between the stretchable insulating film layer having adhesiveness and the stretchable film layer having electrical conductivity is 0.5 N/cm or more.

5. The stretchable laminated sheet according to claim 1, wherein
the stretchable film layer having electrical conductivity includes a conductive film including conductive fine particles and a binder resin.

6. The stretchable laminated sheet according to claim 1, wherein
the binder resin has an elastic modulus of 1 GPa or less and
the binder resin has a rupture elongation of 200% or more.

7. A method for producing the stretchable laminated sheet according to claim 1, comprising
forming the stretchable film layer having electrical conductivity by a coating method or a printing method.

8. A biological information measuring garment comprising
a plurality of body contact components including the stretchable laminated sheet according to claim 1.
